(19) United States
(12) Patent Application Publication
(10) Pub. No.: US 2003/0091662 A1
(43) Pub. Date: May 15, 2003

(54) Apparatus, method and composition for repelling animals

(76) Inventor: Ruth Lipman, New Paltz, NY (US)

Correspondence Address: ARLEN L. OLSEN, SCHMEISER, OLSEN & WATTS, 3 LEAR JET LANE, SUITE 201, LATHAM, NY, 12110, US

(21) Appl. No.: 10/035,305

(22) Filed: Nov. 9, 2001

(51) Int. Cl. A61K 35/78

(52) U.S. Cl. 424/754

(57) ABSTRACT

An apparatus, method and composition for repelling animals are disclosed. The apparatus comprises a layer extracted from a plant of an Alliaceae family on a substrate, wherein the substrate is selected from the group consisting of plants, living animals and manufactured objects and combinations thereof. The method comprises extracting a plant of an Alliaceae family, Allium genus; and applying the extract to an exposed surface of a substrate, wherein the substrate is selected from the group consisting of plants, living animals, manufactured objects and combinations thereof. The composition comprises an extract of a plant of an Alliaceae family, Allium genus; and at least one egg, having a yolk portion.

### BACKGROUND OF THE INVENTION

1. Technical Field

This invention relates generally to a an apparatus, a method and a composition for repelling an animal from an object, comprising: extracting a plant from the family Alliaceae, genus Allium and applying the extract to the object. More particularly, this invention relates to a an apparatus, a method, and a composition for repelling the animal from the object comprising: extracting ramp, onions, chives, shallots, scallions, leeks, wild leek, garlic, garlic chives, wild garlic, ransoms and combinations thereof, and applying the extract to the object.

2. Background Art

Considerable property in the form of plants, containers, insulated wires, cables and the like may be damaged each year by animals that may gnaw or burrow into the property in search of food. Populations of the animals have increased in recent years. Specifically, deer populations in residential communities may forage on landscaped gardens containing wild or cultivated plants, that may result in costly damage to the plants. Techniques to prevent animal damage involving poisons, trapping or glue boards have disadvantages that they may be expensive or harmful to the animals.

Protecting property such as wild or cultivated plants by treating them with extracts derived from other plants may be more environmentally safe because the extracts are naturally occurring and do not result in killing the troublesome animal. In 1899, in U.S. Pat. No. 631,738, Dowie et al. discloses use of chilli pepper and hellebore sprinkled on the property that may be damaged by rats to protect the property. In U.S. Pat. No. 5,985,010, Etscorn, et al. discloses methods for extracting an active repellent ingredient from pepper plants, and applying the extract to the property to be protected, or mixing the active ingredient with caulks, paints, glues or rubber coating materials. Jarrett (aka Colavito), in U.S. Pat. No. 6,117,428, and Colavito (aka Jarett), in U.S. Pat. No. 5,738,851 disclose ruminant repellent compositions derived from plants in the Amarylidaceae family. However, the extracts may pose risk of for example irritation to the people handling them because the extract may be many times more irritating to humans than the pepper plants from which the extract is derived.

There is a need for a repellant that may be used to protect property from damage from animals without posing as much risk of irritation for the humans handling it.

### SUMMARY OF THE INVENTION

The present invention provides a method, comprising:

extracting a plant from the family Alliaceae, genus Allium; and

applying the extract to an exposed surface of a substrate, wherein the substrate is selected from the group consisting of plants, living animals, inanimate objects and combinations thereof.

A second embodiment of the present invention provides an apparatus, comprising:

a substrate having an exposed surface, wherein the substrate is selected from the group consisting of plants, living animals, inanimate objects and combinations thereof;

an extract of a plant from the family Alliaceae, genus Allium, wherein the extract has been applied to the exposed surface.

A third embodiment of the present invention provides a composition, comprising:

an extract of plant from the family Alliaceae, genus Allium; and

at least one egg having a yolk remaining portion.

### BRIEF DESCRIPTION OF THE DRAWINGS

FIG. 1 depicts a front cross-sectional view of an apparatus for extracting plant from the family Alliaceae, genus Allium, according to embodiments of the present invention;

FIG. 2 depicts a method for extracting plant from the family Alliaceae, genus Allium, according to embodiments of the present invention; and

FIG. 3 depicts a front cross-sectional view of an apparatus having a substrate coated with an extract, according to embodiments of the present invention.

### DETAILED DESCRIPTION OF THE PREFERRED EMBODIMENTS

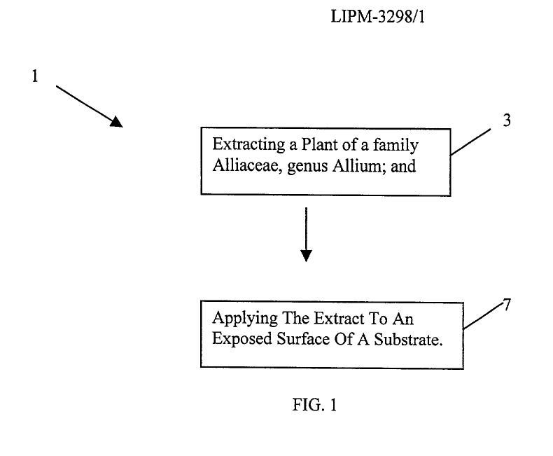

FIG. 1 depicts a method 1 for repelling an animal, comprising: step 3, extracting plant from the family Alliaceae, genus Allium; and step 7, applying the extract to an exposed surface of a substrate. The plant from the family Alliaceae, genus Allium may generally be selected from the group consisting of Allium Cepa (Linnaeus), Allium Ursinum (Linnaeus), Allium Sativum (Linnaeus): Bulbus. The Allium species may be selected from generally from the onion family Alliaceae, genus Allium, consisting of onion hybrids such as Allium wakegi, Allium vineale (Linnaeus, “wild garlic” or ransoms); Allium ampeloprasum (leek); Allium fistulosum (“negi-Japan”, “cong-China”, “Welch onion”); Allium odorum (“Chinese chives”); Allium chinense (Rakkyo, macrostemon, bakeri, are synonyms); Allium scorodoprasum (“Sand leek”); Allium ascalonicum (shallot); Allium tuberosum (“garlic chives”); Allium tricoccum (“ramp or wild leek”); Allium triquetrum (“three-cornered leek”); Allium schoenoprasum (chive). The Allium species may be further selected from the group consisting of ramp, onions, chives, shallots, scallions, leeks, wild leek, garlic, garlic chives, wild garlic or ransoms and combinations thereof.

The Allium cepa (onion) species may be selected from a variety consisting of White Lisbon, Southport White Globe, Southport Red Globe, White Spanish Bunching, White Knight, Red Creole, Red Burgundy, Red Grano, Red Granex and combinations thereof. Bulbing onions are classified into three categories: short, intermediate, and long day types. Short day onions are mild, soft fleshed and cannot be stored for long periods. These types are grown south of 35° latitude and require 12 to 13 hours of light to initialize bulbing. Some short day cultivars include ‘Awahia’, ‘Red Granex’, ‘Red Creole’, ‘White Granex’, ‘Crystal Wax’, ‘Grano’, ‘Granex’, and ‘Yellow Creole’. Long day onions require over 14½ hours of daylight to bulb and are very pungent, hard, and store well. Long day cultivars include ‘Australian Brown’, ‘Carmen’, ‘Southport Red Globe’, ‘Southport White Globe’, ‘White Lisbon’, ‘Ivory’, ‘Fiesta’, ‘Sweet Spanish’, ‘Autumn Spice’, and ‘Downing Yellow Globe’. Intermediate onions are also fleshy and used primarily in fresh market produce and require 13½ to 14 hours of daylight. ‘Cochise Brown’, ‘Stockton Early Red’, ‘Fresno White’, and ‘Rialto’ are common intermediate cultivars.

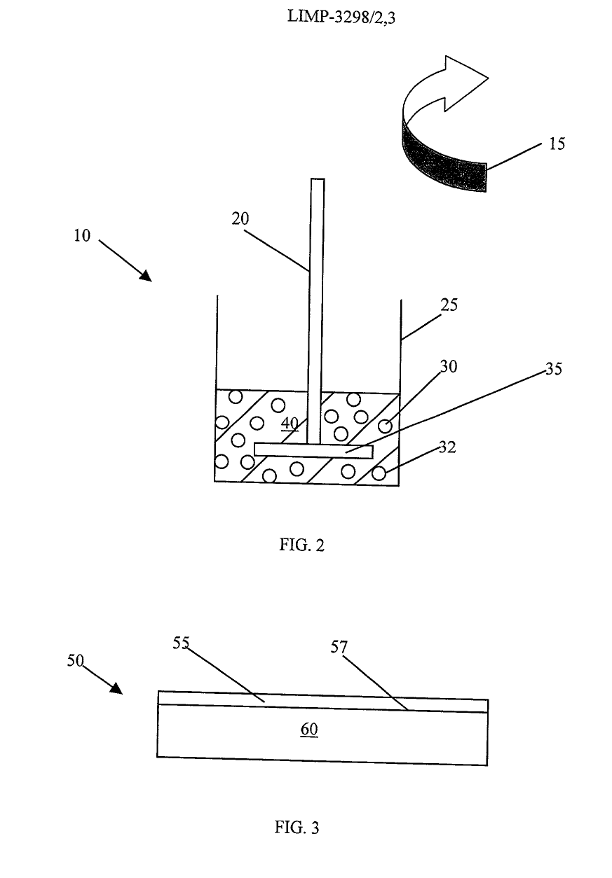

FIG. 2 depicts a front cross-sectional view of an apparatus 10 for extracting a plant from the family Alliaceae, genus Allium, in accordance with the step 3 of the method 1 depicted in FIG. 1 and described in associated text. The apparatus 10 comprises: a container 25; an extract 40 that includes an extracting material and an effective concentration of a material 30 from the family Alliaceae, genus Allium having an exposed surface 32, and a mixing blade 35, operably attached to a shaft 20. Hereinafter, the effective concentration of the extract 40 is an effective weight in grams of a material derived from the family Alliaceae, genus Allium per gallon of the extract 40 that may be applied to a surface of from about 1 to about 1,000 square feet of substrate, such that an animal browsing for food such as a gnawing or chewing ruminant such as, for example, squirrels, rodents, rabbits, chipmunk, beaver, and hooved animals such as deer, horses, or moose will be repelled from the substrate. Alternatively the repelled animal may be a slug (larvae) or insect. The substrate may be, for example, any plant, living animal, manufactured object and combinations thereof. Alternatively, the substrate may be, for example, ornamental plants such as hasta, lilly, gerber daisy, azalea or any of the plants listed in Table 2. Alternatively, the substrate may be cable insulation, caulks, paints, glues or rubber coating materials.

Referring to FIG. 2, the extracting material may be selected from the group consisting of gelatin, water, polyethylene glycol with an average molecular weight ranging from 300 to 600, fatty acid esters of polyglycerines, lecithins, silicone oils, sorbitan fatty acid esters, sorbates of fatty acids, polysorbates of fatty acids, waxes, polyglycerines, triglycerides, fatty acids, fatty oils, paraffins, cooking oils and mixtures thereof.

Referring to FIG. 2, in an embodiment of the present invention, an extract 40 may further comprise an effective amount of a fungicide such as, for example, thiram, wherein the effective amount is from about 0.1 to about 5.9 percent by volume based on a total volume of the extract 40.

Referring to FIG. 2, in an alternative embodiment of the present invention, the apparatus 10 comprises: the container 25; an extract 40 that includes an extracting material and an effective concentration of a juice of a plant from the family Alliaceae, genus Allium, and the mixing blade 35, operably attached to the shaft 20. Hereinafter, the effective concentration of the juice of the plant from the family Alliaceae, genus Allium in the extract 40 may be from about 24 g to about 131 g per gallon of the extract 40.

In a lab study reported by Calvey et al., 26 g of sliced onion yielded 15 ml of juice. See Calvey et al., “Allium Chemistry: Supercritical Fluid Extraction and LC-APCI-MS of Thiosulfinates and Related Compounds from Homogenates of Garlic, Onion, and Ramp. Identification in Garlic and Ramp and Synthesis of 1-Propanesulfinothioic Acid S-Allyl Ester,” J. Agric. Food Chem., 45 (11), 4406-4413, 1997. The sliced onion was juiced with an Oster juice extractor (Milwaukee, Wis.). Alternatively, whole cloves of garlic (2-4 g) or ramp bulbs (2-5 g) were homogenized in 10 mL/g of water at ambient temperature by using a Tissumizer (Teckmar, Cincinnati, Ohio). The solutions were allowed to stand at room temperature for 5-10 min. to ensure complete enzymatic conversion to a mixture of thiosulfinates and other sulfur compounds. Further, Calvey et al. identified the thiosulfinates and related compounds having odors associated with onion and garlic that are formed enzymatically from odorless precursors when the plants of fresh onion (Allium cepa Linnaeus), garlic (Allium sativum Linnaeus), ramp and other Allium species are comminuted, chopped, cut, crushed or juiced and allowed to stand at room temperature for 5-10 min.

Block et al. reported alliinase enzymes catalyze the formation of the thiosulfinates and related compounds having odors associated with onion and garlic. See E. Block, “Allium Chemistry: Synthesis of 1-[Alk(en)ylsulfinyl]propyl Alk(en)yl Disulfides (Cepaenes), Antithrombotic Flavorants from Homogenates of Onion (Allium cepa),” J. Agric. Food Chem., 45 (11), 4414-4422, 1997. Specifically, Block reported the Alliinase enzymes act on S-alk(en)yl cysteine S-oxides giving sulfenic acids, which rearrange to onion lachrymatory factor (LF; C₂H₅CH═S⁺—O⁻ or its tautomer (CH₃CH═CHSOH) or couple affording thiosulfinates, zwiebelanes, or bissulfine. Calvey et al. reported the major thiosulfinates found in extracts of Allium species are methanesulfinothioic acid S-methyl ester; methanesulfinothioic acid S-2-propenyl ester; 2-propene-1-sulfinothioic acid S-methyl ester; methanesulfinothioic acid S-(E)-1-propenyl ester; methanesulfinothioic acid S-(Z)-1-propenyl ester; 1-propanesulfinothioic acid S-methyl ester; methanesulfinothioic acid S-n-propyl ester; (E)-1-propenesulfinothioic acid S-methyl ester; 2-propene-1-sulfinothioic acid S-2-propenyl ester (allicin); 1-propanesulfinothioic acid S-2-propenyl ester; 2-propene-1-sulfinothioic acid S-n-propyl ester; 2-propene-1-sulfinothioic acid S-(E)-1-propenyl ester; 2-propene-1-sulfinothioic acid S-(Z)-1-propenyl ester; (E)-1-propenesulfinothioic acid S-2-propenyl ester; 1-propanesulfinothioic acid S-(E)-1-propenyl ester; propanesulfinothioic acid S-(Z)-1-propenyl ester; (E)-1-propenesulfinothioic acid S-n-propyl ester; propanesulfinothioic acid S-n-propyl ester.

Calvey et al. reported the major sulfur constituents in a supercritical fluid (SF) extract of onion, as determined by cryogenic GC-MS, are methanesulfinothioic acid S-(E,Z)-1-propenyl ester, (E)-1-propenesulfinothioic acid S-methyl ester, (1α,2α,3α,4α,5β)-2,3-dimethyl-5,6-dithiabicyclo[2.1.1]hexane 5-oxide (cis-zwiebelane, (±)-(1α,2α,3α,4α,5β)-2,3-dimethyl-5,6-dithiabicyclo[2.1.1]hexane 5-oxide (trans-zwiebelane, and (E,Z)-propanethial S-oxide (lachrymatory factor, LF).

The following is a list of the bissulfine, zwiebelanes, cepaenes, and ajoene found in extracts of Allium species: (Z,Z)-d,l-2,3-dimethyl-1,4-butanedithial S,S′-dioxide (bissulfine); (1α,2α,3α,4α,5β)-2,3-dimethyl-5,6-dithiabicyclo[2.1.1]hexane 5-oxide (cis-zwiebelane); (±)-(1α,2α,3α,4α,5β)-2,3-dimethyl-5,6-dithiabicyclo[2.1.1]hexane 5-oxide (trans-zwiebelane); methyl 1-(methylsulfinyl)propyl disulfide; methyl (E)-1-(1-propenylsulfinyl)propyl disulfide; 1-(methylsulfinyl)propyl (E,Z)-1-propenyl disulfide; (E)-1-propenyl 1-(1-propenylsulfinyl)propyl disulfide; (E)-1-(1-propenylsulfinyl)propyl propyl disulfide; (E)-1-propenyl 1-(propylsulfinyl)propyl disulfide; (E,Z)-4,5,9-trithiadodeca-1,6,11-triene 9-oxide ((E,Z)-ajoene). Calvey et al. disclosed that in onion, the majority of the 1-propenyl group generated as 1-propenesulfenic acid forms the lachrymatory factor, LF. Calvey et al. further reported (Z,Z)-d,l-2,3-Dimethyl-1,4-butanedithial S,S′-dioxide has previously been isolated from organic extracts of onion. In addition, LC-MS data indicated that cepaene extraction/formation in SC—CO₂ at 35° C. is rapid and that a series of cepaenes [RS(O)CHEtSSR′] are present in an SF extract of onion juice. Ajoene is structurally related to the cepaenes [RS(O)CHEtSSR′] found in onion extracts. Additionally, Calvey et al. reported the zwiebelanes are major sulfur containing compounds found in onion extracts.

Calvey et al. further reported the relative amounts of various thiosulfinates, based on percent peak areas from the TIC, were different in the garlic and ramp extracts. The major thiosulfinate in garlic extracts was allicin (diallyl disulphide oxide 53% of calculated peak areas for total thiosulfinates) with significant contributions from the mixed allyl methyl thiosulfinates (18%), the mixed methyl 1-propenyl thiosulfinates (7%) and the mixed allyl 1-propenyl thiosulfinates (19%). In the frozen ramp extract the relative abundance (RA) of allicin (26%) was less than that found in typical garlic extracts, while the RAs of dimethyl thiosulfinate (15%) and mixed methyl 1-propenyl thiosulfinates (25%) were significantly higher. Botanically, ramp is closely related to European and Asian varieties of wild garlic (Allium ursinum and Allium victorialis; personal communication, Dr. Andrée Nault, 1996). The amount of allicin relative to the other thiosulfinates found in the frozen ramp extract was similar to those reported for wild garlic although the 1-propenyl functionality was more abundant in the former than in the latter. Calvey et al. reported the concentration of the thiosulfinates in onion juice is 100-fold less than that found in extracts of garlic homogenates.

Ramp extracts also appear to contain cepaenes, some of which have MH⁺ ions and retention times consistent with the methyl 1-(methylsulfinyl)propyl disulfide (22), methyl (E,Z)-1-(1-propenylsulfinyl)propyl disulfide (23), and 1-(methylsulfinyl)propyl (E,Z)-1-propenyl disulfide isomers. Other components, which may be particular cepaenes, had retention times that differed from those observed for the cepaenes found in onion extracts or for the synthetic standards analyzed. Their chromatographic retention times suggested the incorporation of the allyl group into the cepaene backbone. Calvey et al. reported the formation of allyl containing cepaenes in extracts of homogenates prepared from combined onion and garlic. Other late eluting constituents observed in garlic and ramp extracts by LC with UV detection were not readily observed by LC-MS in the TIC. These components have retention times and UV spectra characteristic of the dithiins, alkyl sulfides, and polysulfides.

The data for a late eluting constituent (t_{R} 19-20 min) in garlic extracts were consistent with data for ajoene [AllS(O)CH₂═CHSSAll, where “All” represents an allyl organic group], a major component found in oil-macerated garlic products. Although ajoene was not conclusively identified in ramp extracts, there is some evidence that ajoene-related compounds, where methyl functionalities are substituted for the allyl groups [MeS(O)CH₂CH═CHSSMe, (E,Z)-2,3,7-trithiaoct-4-ene 7-oxide], are present in SF extracts of ramp homogenates.

In summary, Calvey et al. and Block et al. disclosed that Block et al. reported alliinase enzymes catalyze the formation of the thiosulfinates and related compounds having odors associated with onion and garlic. Specifically, Block reported the alliinase enzymes act on S-alk(en)yl cysteine S-oxides giving sulfenic acids from comminuted, chopped, crushed or juiced plants belonging to the family Alliaceae, genus Allium that have been allowed to stand at room temperature for 5-10 min. Block further reported that the alliinase enzymes catalyze rearrangement of sulfenic acids to onion lachrymatory factor (LF; C₂H₅CH═S⁺—O⁻ or its tautomer (CH₃CH═CHSOH) or couple sulfenic acids, affording thiosulfinates, zwiebelanes, or bissulfine.

FIG. 2 depicts a front cross-sectional view of an apparatus 10 for extracting a plant from the family Alliaceae, genus Allium, in accordance with the step 3 of the method 1 depicted in FIG. 1 and described in associated text. The apparatus 10 comprises: a container 25; an extract 40 that includes an extracting material and an effective concentration of a comminuted, crushed or chopped plant 30 of a plant from the family Alliaceae, genus Allium having an exposed surface 32, and a mixing blade 35, operably attached to a shaft 20. Hereinafter, the effective concentration of the comminuted, crushed or chopped plant 30 from the family Alliaceae, genus Allium in the extract 40 may be from about 41 g to about 227 g per gallon of the extract 40.

Referring to FIG. 2 and the lab study of Calvey et al., the following thiosulfinates may be formed in the extract 40 when the plant from the family Alliaceae, genus Allium is extracted according to the step 3 of the method and allowed to stand at room temperature for 5-10 min.: methanesulfinothioic acid S-methyl ester; methanesulfinothioic acid S-2-propenyl ester; 2-propene-1-sulfinothioic acid S-methyl ester; methanesulfinothioic acid S-(E)-1-propenyl ester; methanesulfinothioic acid S-(Z)-1-propenyl ester; 1-propanesulfinothioic acid S-methyl ester; methanesulfinothioic acid S-n-propyl ester; (E)-1-propenesulfinothioic acid S-methyl ester; 2-propene-1-sulfinothioic acid S-2-propenyl ester (allicin); 1-propanesulfinothioic acid S-2-propenyl ester; 2-propene-1-sulfinothioic acid S-n-propyl ester; 2-propene-1-sulfinothioic acid S-(E)-1-propenyl ester; 2-propene-1-sulfinothioic acid S-(Z)-1-propenyl ester; (E)-1-propenesulfinothioic acid S-2-propenyl ester; 1-propanesulfinothioic acid S-(E)-1-propenyl ester; propanesulfinothioic acid S-(Z)-1-propenyl ester; (E)-1-propenesulfinothioic acid S-n-propyl ester; propanesulfinothioic acid S-n-propyl ester.

In addition, referring to FIG. 2 and the lab study by Calvey et al., the following bissulfines, zwiebelanes, cepaenes, and ajoene may be formed in the extract 40 when the plant from the family Alliaceae, genus Allium is extracted according to the step 3 of the method 1 and allowed to stand at room temperature for from about 5 to about 10 min.: (Z,Z)-d,l-2,3-dimethyl-1,4-butanedithial S,S′-dioxide (bissulfine); (1α,2α,3α,4α,5β)-2,3-dimethyl-5,6-dithiabicyclo[2.1.1]hexane 5-oxide (cis-zwiebelane); (±)-(1α,2α,3α,4α,5β)-2,3-dimethyl-5,6-dithiabicyclo[2.1.1]hexane 5-oxide (trans-zwiebelane); methyl 1-(methylsulfinyl)propyl disulfide; methyl (E)-1-(1-propenylsulfinyl)propyl disulfide; 1-(methylsulfinyl)propyl (E,Z)-1-propenyl disulfide; (E)-1-propenyl 1-(1-propenylsulfinyl)propyl disulfide; (E)-1-(1-propenylsulfinyl)propyl propyl disulfide; (E)-1-propenyl 1-(propylsulfinyl)propyl disulfide; (E,Z)-4,5,9-trithiadodeca-1,6,11-triene 9-oxide ((E,Z)-ajoene).

Referring to FIG. 2, in an alternative embodiment of the present invention, an effective concentration of a powder of a plant from the family Alliaceae, genus Allium and may be added to the extract 40 of the apparatus 10, in accordance with the step 3 of the method 1, depicted in FIG. 1 and described in associated text, instead of adding a comminuted, crushed or chopped plant 30. The apparatus 10 comprises: a container 25; an extract 40 that includes an extracting material; an effective concentration of a powder from the family Alliaceae, genus Allium, having an exposed surface 32; and a mixing blade 35, operably attached to a shaft 20. Hereinafter, the effective concentration of the powder from the family Alliaceae, genus Allium in the extract 40 may be from about 0.1 tablespoons to about 10 tablespoons per gallon of the extract 40.

The powder 30 in the extract 40 may be obtained by grinding or pulverizing fresh onions in a mortar and pastel or other appropriate pulverizing device known in the art that have been dehydrated according to Aebi et al, in U.S. Pat. No. 5,368,873, herein incorporated by reference. Fresh onions (Creole variety, 18% solids were sliced, soaked in a 40% sucrose solution and dehydrated according to the method of Aebi et al. Excess sucrose was removed by centrifugation. Lastly, the onions were dehydrated with heat. Aebi et al. disclosed that when 1000 g of fresh creole onion of the family Alliaceae, genus Allium was dehydrated, the dehydrated product weighed 245 g. The rehydrated onion exhibited pungency, heat and flavor similar in character to fresh onion.

In summary, Aebi et al. disclose a method for dehydrating fresh sliced onions, wherein the dehydrated product exhibited pungency heat and favor similar in character to fresh onions when it was rehydrated.

The powder 30 may alternatively be obtained by a freeze drying process. In the freeze drying process, a first step may be to allow the comminuted, crushed or chopped plant 30 of a plant from the family Alliaceae, genus Allium to remain at room temperature for at least 5-10 min. The sulfenic acids reported by Block may be produced enzymatically by the action of the alliinase enzymes on S-alk(en)yl cysteine S-oxides. In addition, alliinase enzymes catalyze the rearrangement of the sulfenic acids, also reported by Block, to onion lachrymatory factor (LF; C₂H₅CH═S⁺—O⁻ or its tautomer (CH₃CH═CHSOH) or the coupling of the sulfenic acids, also reported by Block, to the odor and flavor full thiosulfinates, zwiebelanes, or bissulfine may occur in the comminuted, crushed or chopped plant 30. In a second step of the freeze drying process, the plant 30 may be subjected to an operating temperature from about room temperature to about −25° C. and a reduced pressure provided by a vacuum pump or water aspirator. At the operating vacuum pressure of the freeze-drying process water cannot exist as a liquid. It exists as ice or vapor and sublimates directly from ice to vapor. As temperatures are increased at a controlled rate the water in the plant 30 sublimates directly to vapor as it leaves the plant 30. Refrigeration panels within the freeze-drying chamber operate at a temperature around −25 degrees Celsius. As vapor is forced off the plant 30 by increasing temperatures, the vapor is drawn toward the colder refrigeration panels where it is converted back to solid form through deposition. A balanced sublimation-deposition process maintains proper vacuum in the chamber.

Careful control of pressure and temperature are essential to the freeze-drying process. If temperatures are increased too quickly, the refrigeration panels may not keep up with the vapor-generating sublimation process and pressure in the chamber may raise thereby allowing liquid water to form in the product. This can be avoided by carefully controlling the rate of temperature increase.

Once the plant 30 has been properly freeze-dried, it may be converted into powder 30 by grinding in a mortar with a pestel, or other appropriate grinding device known in the art.

Alternatively, the powder 30 may be obtained by the freeze drying process from the plant 30 without first allowing the comminuted, crushed or chopped plant 30 to stand at room temperature for from 5 to 10 min. or at elevated temperature in order to avoid the reaction of sulfenic acids such as alliin with alliinase to produce allicin (diallyl thiosulfinate). Plummer, in U.S. Pat. No. 5,705,152, herein incorporated by reference, disclosed a method for freeze drying whole garlic cloves to produce garlic granules or powder, for use as a antimicrobial material. The freeze drying process entailed the rapid freezing of the substantially whole cloves followed by gently drying at temperatures between −25 and −5° C.

Plummer disclosed whole garlic cloves were peeled to remove the brittle outer skin. The clove was left entirely intact (cutting the scar causes some allicin to be produced). The cloves were then frozen to −30° C. Primary freeze drying took place with the product at temperatures between −25° C. and −5° C.

Plummer stated when the primary freeze drying phase was completed, the temperature of the garlic cloves rose rapidly to +20° C. where it remained whilst secondary drying took place to remove desorbed water. This took approximately 2 hours, the entire drying process taking between 12 and 24 hours.

Plummer disclosed the resulting freeze-dried powder contained 1.55% alliin and had allicin potency (as measured by the size of zone of inhibition of the yeast Candida Albicans) of 15 mm.

Plummer disclosed similar results, but with slightly less allicin potency, were obtained by cutting off the scar at the end of each clove to accelerate moisture loss during the freeze-drying process.

Plummer disclosed similar results were obtained by cryogenically crushing, dicing or milling of the frozen whole cloves, reaction between alliin and alliinase being substantially prevented by ensuring that the temperatures did not exceed −20° C. at any point during the cryogenic treatment.

Plummer disclosed the results achieved using freeze-dried whole garlic cloves are compared with those from crushed garlic in the following table.

TABLE 1
Percent by weight Alliin from freeze-dried whole garlic
cloves and freeze-dried crushed garlic disclosed by Plummer.

| | Alliin | Allicin |
|---|---|---|
| Whole Cloves Freeze-Dried | 1.55% | 15 |
| (According to The Invention) | | |
| Crushed Garlic 4° C. | 0.42% | 13 |
| 30 Mins Freeze-Dried | | |
| Crushed Garlic 4° C. | 0.11% | 12 |
| 2 Hours Freeze-Dried | | |
| Crushed Garlic 4° C. | Trace | 9.5 |
| 24 Hours Freeze-Dried | | |
| Crushed Garlic 20° C. | 0.0411 | 11 |
| 30 Mins Freeze-Dried | | |
| Crushed Garlic 20° C. | Trace | 10 |
| 2 Hours Freeze-Dried | | |
| Crushed Garlic 20° C. | Trace | 6 |
| 24 Hours Freeze-Dried | | |

Plummer disclosed the above table demonstrates the rapid reduction in the level of alliin after disintegration of a garlic clove, and the concomitant decrease on the allicin potency as demonstrated by a bioassay method against the yeast Candida albicans.

Plummer disclosed the results show that allicin is rapidly produced from alliin and alliinase even at low temperatures. Also the antimicrobial activity of the preparation is decreased after allicin is formed and this decrease is dependent both upon time and temperature.

Plummer disclosed in the extreme cases above, garlic cloves which were crushed and kept for 24 hours at 20° C. before freeze drying had only 40% of the activity of garlic which was freeze-dried as the whole clove.

In summary, referring to the results in Table 1, disclosed by Plummer, the powder obtained from the clove left entirely intact (cutting the scar causes some allicin to be produced). and then frozen to −30° C. had 1.55 weight percent alliin based on weight of the powder. Plummer further disclosed that alliin is a non-odiferous derviative of the amino acid cysteine.

Referring to FIG. 2, in all embodiment of the present invention, the extract 40 may further comprise from about 0.5-2.0 percent by volume of the egg having a yolk portion, wherein the egg may be, for example, a chicken egg. The egg yolk portion may emulsify the thiosulfinates, bissulfines, zwiebelanes, cepaenes, and ajoene in the extract 40, resulting in the extract 40 becoming homogeneous. Alternatively, instead of egg or egg yolk, the extract 40 may further comprise lecithin or phospholipids such as, for example, phosphatidylcholine (PC). An effective amount of lecithin in the extract 40 is from about 0.08-0.33 percent by volume. An effective amount of phosphatidylcholine (PC) in the extract 40 is from about 0.02-0.09 percent by volume.

Lecithin or phosphatidylcholine (PC) may be obtained from for example egg yolk by a method employing extraction and centrifugation, according to Merkle et al., in U.S. Pat. No. 6,217,926, herein incorporated by reference. In general, according to Merkle et al., the yolk of various strains of chicken eggs is 30% to 36% lipid comprising about 65% triglycerides, 28.3% phospholipids and 5.2% cholesterol.

Milne et al., in U.S. Pat. No. 5,958,463, herein incorporated by reference, further disclosed phosphatidylcholine (PC) may be extracted and purified generally from animal lecithin, plant lecithin such as, for example, from soy, and from egg lecithin. Further, Milne et al. disclosed that the PC is the material in plant lecithin that actually does the encapsulating in the liposomal microencapsulation process. According to Milne, a molecule of PC has a phosphate head with a choline moiety and some fatty acid chains that form a tail portion. The fatty acid chains are nonpolar and therefore repel water. The phosphate head of the PC molecule attracts water. When placed in water, the molecules coalesce so that the molecule tails are directed one way and the heads another to produce the vesicle formation of the liposomal encapsulation technique.

According to Milne et al., a predetermined lecithin stock solution is produced and mixed with an amount of a preselected active agent such as, for example, an agricultural pesticide. The resulting active agent solution is mixed with water forming an agricultural liquid formulation having the active agent encapsulated in a liposomal composition.

Referring to Milne et al., the active agent solution is directed to a slow release pesticide composite formulation comprising a pesticide material in an amount sufficient to control pests which have a deleterious effect on agricultural crops.

In summary, Milne et al. disclose that lecithin or PC extracted from lecithin may be used to emulsify the chemicals such as pesticides in water solutions, but Milne et al. do not disclose addition of egg having a yolk or egg yolk alone to emulsify chemicals in water, nor does Milne et al. disclose that lecithin or PC extracted from lecithin may be used to emulsify thiosulfinates, bissulfines, zwiebelanes, cepaenes, and ajoene in the extract 40, resulting in the extract 40 becoming homogeneous.

Todd, in U.S. Pat. No. 6,013,304, herein incorporated by reference, disclosed extracting the powder or flake obtained by dehydration of a fresh plant onion powder, such as for example, Gilroy Foods, Inc., 92700 Toasted Special Onion Powder, using an edible solvent such as for example vegetable oils, lecithin and animal fats and a pressing process. Todd disclosed the resulting extract had an aroma and flavor profile characteristic of onion that was four (4) times the strength of the starting powder.

Referring to FIG. 2, in another embodiment of the present invention, the apparatus 10 may be used for extracting an extract of the family Alliaceae, genus Allium, in accordance with the step 3 of the method 1 depicted in FIG. 1 and described in associated text. The extract of the family Alliaceae, genus Allium may be extracted from the powder or flake obtained by dehydration of a fresh plant onion powder, according to the method of Todd. The apparatus 10 comprises: a container 25; an extract 40 that includes an extracting material; an egg having a concentration from about 0.5 volume percent to about 2.0 volume percent based on a total volume of the extract 40; an effective concentration of the extract of the powder or flake obtained by dehydration of a fresh plant onion powder, instead of the effective concentration of the comminuted, crushed or chopped plant 30. Hereinafter, the effective concentration of the extract of the powder or flake obtained by dehydration of a fresh plant onion powder may be not less than 25% of the effective concentration of the powder of the family Alliaceae, genus Allium. The effective concentration of the extract of the powder or flake obtained by dehydration of a fresh plant onion powder may be not less than from about 0.025 tablespoons to about 2.5 tablespoons per gallon of the extract 40.

FIG. 3 depicts a front cross-sectional view of an apparatus 50, comprising a substrate 60, wherein an extract layer 55 has been formed by application of the extract 40 to an exposed surface 57 of the substrate 60. Referring to FIGS. 2-3, when a gallon of the effective concentration of the plant from the family Alliaceae, genus Allium in the extract 40 is applied to the exposed surface 57 of the substrate 60, the extract layer 55 may be formed. The substrate 60 is selected from the group consisting of plants, living animals, inanimate objects and combinations thereof. Applying the effective concentration of the extract 40 to the exposed surface 57 of the substrate 60 forms the extract layer 55 on the exposed surface 57 and may repel an animal away from the substrate 60.

The substrate 60 may be selected from any of the plants 60 listed as follows in Table 2:

TABLE 2
Examples of the Substrate 60, wherein the substrate 60 is a plant.

| Botanical Name | Common Name | Botanical Name | Common Name |
|---|---|---|---|
| Anchimenes sp. | Pansy | Helianthus annus | Sunflower |
| Endymion sp. | Wood Hyacinth | Geranium | Cranesbill |
| | | maculatim | Geranium |
| Hedera helix | English Ivy | Iris sp. | Iris |
| Paeonia sp. | Peony | Rudbeckia sp. | Coneflower |
| Sedum purpureum | Autumn Joy | Thalictrum sp. | Meadow Rue |
| ‘Autumn Joy’ | Sedum | | |
| Amelanchier | Downy | Amelanchier | Allegheny |
| arborea | Serviceberry | laevis | Serviceberry |
| Campsis radicans | Trumper | Chaenomeles | Japanese |
| | Creeper | speciosa | Flowering |
| | | | Quince |
| Cornus racemosa | Panicled | Cotinus | Smokebush |
| | Dogwood | coggygria | |
| Cotoneaster spp. | Cotoneaster | Forsythia (x) | Border |
| | | intermedia | Forsythia |
| Hybiscus | Rose of Sharon | Hydrangea | Smooth |
| | | arborescens | Hydrangea |
| Hydrangea anomala | Climbing | Hydrangea | Panicle |
| petiolaris | Hydrangea | paniculata | Hydrangea |
| Ilex crenata | Japanese Holly | Ilex (x) | China Girl/ |
| | | meserveae | Boy Holly |
| Lonicera (x) | Goldflame | Ligustrum spp. | Privet |
| hackrottii | Honeysuckle | | |
| Parathenocissus | Virginia | Philadelphus | Sweet Mock |
| quinquifolia | Creeper | coronarius | Orange |
| Potentilla fruiticosa | Bush | Pyracantha | Firethorn |
| | Cinquefoil | coccinea | |
| Pyrus calleryana | Bradford | Rhododendron | Deciduous |
| ‘Bradford’ | Gallery Pear | spp. | Azaleas |
| Rhododendron | Carolina | Rhododendron | Rosebay |
| carolinianum | Rhododendron | maximum | Rhododendron |
| Rhus typhina | Staghorn | Rosa multiflora | Multiflora Rose |
| | Sumac | | |
| Rosa rugosa | Rugosa Rose | Spirea (x) | Anthony |
| | | bumalda | Waterer Spirea |
| Spirea prunifolia | Bridal Wreath | Syringa (x) | Persian Lilac |
| | Spirea | persica | |
| Syringa reticulata | Japanese Tree | Syringa vilosa | Late Lilac |
| | Lilac | | |
| Viburnum carlesii | Korean Spice | Viburnum | Dbl. File |
| | | plicatum | Viburnum |
| Viburnum | Viburnum | Weigela florida | tometosum |
| rhytidophyllum | Leather Leaf | | Old Fashioned |
| | Viburnum | | Weigela |
| Abeis concolor | White Fir | Acer griseum | Paperbark |
| | | | Maple |
| Acer rubrum | Red Maple | Acer saccarinum | Silver Maple |
| Acer saccharum | Sugar Maple | Aesculus | Common Horse |
| | | hippocastanum | Chestnut |
| Cryptomeria | Japanese Cedar | Hamamelis | Common |
| japonica | | virginiana | Witch Hazel |
| Juniperus virginiana | Eastern Red | Larix decidua | European |
| | Cedar | | Larch |
| Magnolia (x) | Saucer | Metasequoia | Dawn |
| soulangiana | Magnolia | glyptostroboides | Redwood |
| Pinus strobus | Eastern White | Prunus avium | Sweet Cheny |
| | Pine | | |
| Pseudotsuga | Douglas Fir | Pyrus communis | Common Pear |
| menziesii | | | |
| Quercus alba | White Oak | Quercus prinus | Chestnut Oak |
| Quercus rubra | Northern Red | Salix spp. | Willows |
| | Oak | | |
| Tilia cordata | Greenspire | Tilia americana | Basswood |
| ‘Greenspire’ | Littleleaf | | |
| | Linden | | |
| Tsuga canadensis | Eastern | Tsuga caroliniana | Carolina |
| | Hemlock | | Hemlock |
| Alcea rosea | Hollyhocks | Impatiens sp. | Impatiens |
| Tithonia | Mexican | | |
| rotundifolia | Sunflower | | |
| Clematis spp. | Clematis | Crocus sp. | Crocus |
| Hemerocallis sp. | Daylily | Hosta sp. | Hosta |
| Lobelia cardinalis | Cardinal | Phlox sp. | Phlox |
| | Flower | | |
| Tulipa sp. | Tulips | | |
| Cornus mas | Cornelian | Euonymus alatus | Winged |
| | Dogwood | | Euonymus |
| Euonymus fortunei | Winter Creeper | Rhododendron | Rhododendrons |
| | | spp. | |
| Rosa (x) hybrid | Hybrid Tea | Taxus spp. | Yews |
| | Rose | | |
| Thuja occidentalis | American | | |
| | Arborvitae | | |
| Abies balsamea | Balsam Fir | Abies fraseri | Fraser Fir |
| Acer platanoides | Norway Maple | Cercis canadensis | Eastern Redbud |
| Chamaecyparis | Atlantic | Malus spp. | Apples |
| thyoides | White Cedar | | |
| Prunus spp. | Cherries | Prunus spp. | Plums |
| Sorbus aucuparia | European | | |
| | Mountain Ash | | |

### EXAMPLE 1

Referring to FIG. 2, the apparatus 10 may be used for extracting a Red Creole onion bulb of the family Alliaceae, genus Allium, in accordance with the step 3 of the method 1 depicted in FIG. 1 and described in associated text. The apparatus 10 comprises: a container 25; an extract 40 that includes an extracting material; an egg having a concentration from about 0.5 weight percent to about 2.0 volume percent based on a total volume of the extract 40; an effective concentration of a comminuted, crushed or chopped Red Creole onion bulb 30 from the family Alliaceae, genus Allium having an exposed surface 32; and a mixing blade 35, operably attached to a shaft 20. The extract 40 may be obtained by rapidly stirring the mixture of egg, extracting material and the effective concentration of comminuted, crushed or chopped Red Creole onion bulb 30. Hereinafter, the effective concentration of the comminuted, crushed or chopped plant 30 in the extract 40 may be from about 41 g to about 227 g in a gallon of water.

Referring to FIG. 3, the apparatus 50 for repelling plants, living animals, manufactured objects and combinations thereof of the present invention was formed by applying at least one gallon of the extract 40 having the effective concentration of the powder 30 to from about 1 to about 1,000 square feet of the surface 57 of the substrate 60. The application was repeated a maximum of once per two and a half weeks.

Referring to FIG. 3, although untreated substrates 60 such as, for example, ornamental plants such hasta, lilly, gerber daisy and rhododendron, such as for example, azalea were damaged by deer browsing or feeding and roots of grass were damaged by insect larvae such as for example the japanese beetle larvae browsing and feeding and japanese red maples were damaged by japanese beetles browsing and feeding, no damage was observed from deer, japanese beetle larvae or japanese beetles browsing or feeding on the treated substrates 60 of the apparatus 50 from May to September, 2001.

### EXAMPLE 2

Referring to FIG. 2, the apparatus 10 may be used for extracting a White Southport Globe onion bulb of the family Alliaceae, genus Allium, in accordance with the step 3 of the method 1 depicted in FIG. 1 and described in associated text. The apparatus 10 comprises: a container 25; an extract 40 that includes an extracting material; an egg having a concentration from about 0.5 volume percent to about 2.0 volume percent based on a total volume of the extract 40; an effective concentration of a comminuted, crushed or chopped White Southport Globe onion bulb 30 from the family Alliaceae, genus Allium having an exposed surface 32; and a mixing blade 35, operably attached to a shaft 20. The extract 40 may be obtained by rapidly stirring the mixture of egg, extracting material and the effective concentration of comminuted, crushed or chopped White Southport Globe onion bulb 30. Hereinafter, the effective concentration of the comminuted, crushed or chopped plant 30 in the extract 40 may be from about 41 g to about 227 g in a gallon of water.

Referring to FIG. 3, the apparatus 50 for repelling plants, living animals, manufactured objects and combinations thereof of the present invention was formed by applying at least one gallon of the extract 40 having the effective concentration of the powder 30 to from about 1 to about 1,000 square feet of the surface 57 of the substrate 60. The application was repeated a maximum of once per two and a half weeks. Referring to FIG. 3, although untreated substrates 60 such as, for example, ornamental plants such hasta, lilly, gerber daisy and rhododendron, such as for example, azalea were damaged by deer browsing or feeding and roots of grass were damaged by insect larvae such as for example the japanese beetle larvae browsing and feeding and japanese red maples were damaged by japanese beetles browsing and feeding, no damage was observed from deer, japanese beetle larvae or japanese beetles browsing or feeding on the treated substrates 60 of the apparatus 50 from May to September, 2001.

### EXAMPLE 3

Referring to FIG. 2, in another embodiment of the present invention, the apparatus 10 may be used for extracting a powder 30 from a Red Creole onion bulb or a White Southport Globe onion bulb, in accordance with the step 3 of the method 1 depicted in FIG. 1 and described in associated text. The apparatus 10 comprises: a container 25; an extract 40 that includes an extracting material; an egg having a concentration from about 0.5 volume percent to about 2.0 volume percent based on a total volume of the extract 40; an effective concentration of the powder 30 from the Red Creole onion bulb or a White Southport Globe onion bulb, instead of the effective concentration of the comminuted, crushed or chopped plant 30. The extract 40 may be obtained by rapidly stirring the mixture of egg, extracting material and the effective concentration of powder 30 from the Red Creole onion bulb 30 or the White Southport Globe onion bulb 30. Hereinafter, the effective concentration of the powder 30 may be from about 0.1 tablespoons to about 10 tablespoons per gallon of the extract 40.

Referring to FIG. 3, the apparatus 50 for repelling plants, living animals, manufactured objects and combinations thereof of the present invention was formed by applying at least one gallon of the extract 40 having the effective concentration of the powder 30 to from about 1 to about 1,000 square feet of the surface 57 of the substrate 60. The application was repeated a maximum of once per two and a half weeks. Referring to FIG. 3, although untreated substrates 60 such as, for example, ornamental plants such hasta, lilly, gerber daisy and rhododendron, such as for example, azalea were damaged by deer browsing or feeding and roots of grass were damaged by insect larvae such as for example the japanese beetle larvae browsing and feeding and japanese red maples were damaged by japanese beetles browsing and feeding, no damage was observed from deer, japanese beetle larvae or japanese beetles browsing or feeding on the treated substrates 60 of the apparatus 50 from May to September, 2001.

1. A method, comprising:
extracting a plant of an Alliaceae family, Allium genus; and
applying the extract to an exposed surface of a substrate, wherein the substrate is selected from the group consisting of plants, living animals, manufactured objects and combinations thereof.

2. The method of claim 1, wherein the Allium genus includes Allium Cepa.

3. The method of claim 2, wherein the Allium Cepa is selected from the group consisting of White Lisbon, Southport White Globe, Southport Red Globe, White Spanish Bunching, White Knight, Red Creole, Red Burgundy, Red Grano, Red Granex and combinations thereof.

4. The method of claim 1, wherein the Allium genus is selected from the group consisting of Allium Cepa (Linnaeus), Allium Ursinum (Linnaeus), Allium Sativum (Linnaeus): Bulbus.

5. The method of claim 1, wherein the Allium genus includes Allium Sativum.

6. The method of claim 1, wherein the Allium genus is selected from the group consisting of ramp, onions, chives, shallots, scallions, leeks, wild leek, garlic, garlic chives, wild garlic, ransoms and combinations thereof.

7. The method of claim 1, wherein the Allium genus is extracted with a material selected from the group consisting of gelatin, water, polyethylene glycol with an average molecular weight ranging from 300 to 600, fatty acid esters of polyglycerines, lecithins, silicone oils, sorbitan fatty acid esters, sorbates of fatty acids, polysorbates of fatty acids, waxes, polyglycerines, triglycerides, fatty acids, fatty oils, paraffins, cooking oils and mixtures thereof.

8. The method of claim 1, wherein the Allium genus is selected from the group consisting of Allium cepa (onion); Allium niponicum (“nobiru”); Allium sativum (garlic); Allium wakegi (“wakegi”); Allium ursinum (“wild garlic” or ransoms); Allium vineale (“wild garlic” or ransoms); Allium ampeloprasum (leek); Allium fistulosum (“negi” Welch onion); Allium bakeri; Allium ascalonicum (shallot); Allium tuberosum (“garlic chives”); Allium tricoccum (“ramp or wild leek”); Allium triquetrum (“three-cornered leek”); Allium schoenoprasum (chive).

9. The method of claim 1, wherein the extract further comprises alliinase enzyme.

10. The method of claim 1, wherein the extract contains a flavor precursor selected from the group consisting of S-methyl, S-ethyl, S-propyl, S-propenyl and S-allyl cysteine sulfoxides and combinations thereof.

11. The method of claim 1, wherein the extract further comprises at least one egg, having a yolk portion.

12. The method of claim 1, wherein the extract further comprises a preservative.

13. An apparatus, comprising:
a substrate having an exposed surface, wherein the substrate is selected from the group consisting of plants, living animals, manufactured objects and combinations thereof;
an extract of a plant of an Alliaceae family, Allium genus, wherein the extract has been applied to the exposed surface.

14. The apparatus of claim 13, wherein the extract further comprises an egg, having a yolk portion.

15. The apparatus of claim 13, wherein the Allium genus is selected from the group consisting of ramp, onions, chives, shallots, scallions, leeks, wild leek, garlic, garlic chives, wild garlic, ransoms and combinations thereof.

16. The method of claim 13, wherein the Allium genus is extracted with a material selected from the group consisting of gelatin, water, polyethylene glycol with an average molecular weight ranging from 300 to 600, fatty acid esters of polyglycerines, lecithins, silicone oils, sorbitan fatty acid esters, sorbates of fatty acids, polysorbates of fatty acids, waxes, polyglycerines, triglycerides, fatty acids, fatty oils, paraffins, cooking oils and mixtures thereof.

17. The method of claim 13, wherein the Allium genus is selected from the group consisting of Allium cepa (onion); Allium niponicum (“nobiru”); Allium sativum (garlic); Allium wakegi (“wakegi”); Allium vineale (wild garlic); Allium porrum (leek); Allium fistulosum (“negi” Welch onion); Allium bakeri; Allium ascolonicum (shallot); Allium tuberosum (“chinese chives”); Allium tricoccum (“wild leek”); Allium schoenoprasum (chive).

18. The method of claim 13, wherein the extract further comprises an alliinase enzyme.

19. A composition, comprising:
an extract of a plant of an Alliaceae family, Allium genus; and
at least one egg, having a yolk portion.

20. The composition of claim 19, wherein the Allium genus is selected from the group consisting of ramp, onions, chives, shallots, scallions, leeks, wild leek, garlic, garlic chives, wild garlic, ransoms and combinations thereof.
